(19) United States
(12) Patent Application Publication
(10) Pub. No.: US 2025/0195758 A1
(43) Pub. Date: Jun. 19, 2025

(54) SYSTEM AND METHOD FOR ADMINISTERING A BUTTERFLY NEEDLE TO A PATIENT

(71) Applicant: KORU Medical Systems, Inc., Mahwah, NJ (US)

(72) Inventors: GABRIELLA ANNE FERRARA, Ridgewood, NJ (US); BRIAN CHRISTOPHER CASE, Lake Villa, IL (US); THOMAS ALAN ALHEIDT, Sussex, NJ (US); REED DEWINTER, Cincinnati, OH (US); DONALD FRASER VANROYEN, Philadelphia, PA (US)

(73) Assignee: KORU Medical Systems, Inc., Mahwah, NJ (US)

(21) Appl. No.: 18/543,866

(22) Filed: Dec. 18, 2023

(51) Int. Cl. A61M 5/158; A61M 5/42

(52) U.S. Cl. CPC A61M 5/158; A61M 5/422; A61M 2005/1585; A61M 2005/1586; A61M 2005/1587

(57) ABSTRACT

Provided is a system for administering a butterfly needle to a patient. The system includes a butterfly needle insertion device, having: a housing provided by a shell with a distal end and a proximal opening, the proximal opening having two opposing grooves each sized to receive a wing of a butterfly needle, and two opposing channels disposed between the opposing grooves. A drive plunger occupies at least a portion of the space defined by the shell of the housing and in shifting engagement with the housing. The drive plunger has a distal end to be engaged by a stop to hold the drive plunger in a retracted position and a proximal end structured and arranged to receive the butterfly needle with the wings extending through the opposing grooves and beyond the housing. A supply line of the butterfly needle received by one of the opposing channels. A butterfly needle retainer is disposed within the housing to temporarily hold the butterfly needle to the proximal end of the drive plunger when the drive plunger is in the retracted position. A drive spring associated with the drive plunger such that when the drive plunger is driven into the housing to the retracted position, the drive spring is loaded and when the drive plunger is released, the drive plunger travels distally from the force of the drive spring unloading to deliver the butterfly needle into the patient. An associated method of use is provided as well.

### FIELD OF THE INVENTION

The present invention relates generally to systems and methods for subcutaneous infusion systems and equipment, and more particularly to a system for inserting needles into tissue and methods of use.

### BACKGROUND

Infusion systems for the delivery of liquid pharmaceuticals are widely used and relied upon by patients and care givers alike.

With a typical injection, commonly referred to as a “shot” a patient is quickly injected with a medicant provided by a handheld syringe directly connected to a needle. The caregiver punctures the patient's skin with the needle to reach desired tissues or structures, depresses the plunger of the syringe to inject the medicant, and then withdraws the needle. This process is typically performed in less than one minute.

With an infusion, typically the medicant is delivered over a period of time. The needle is inserted to reach the desired tissues or structures and then remains in contact with those tissues or structures for the duration of the treatment which is typically measurable in many minutes or hours.

One form of infusion therapy is Immuno Globulin (Ig) therapy, and it is frequently used to improve the quality of life for patients with conditions such as Primary Immune Deficiency (PID), Secondary Immune Deficiency (SID), Chronic Inflammatory Demyelinating Polyradiculoneuropathy (CIPD), and Severe Combined Immunodeficiency (SCID). Historically, Ig therapy has been administered intravenously (IVIg) every 3-4 weeks, which can result in inconsistent serum levels and burdensome infusion experiences for patients.

Subcutaneous Ig therapy (SCIg) is a more convenient option that allows for consistent serum levels and can be done at home. However, SCIg has its own challenges, such as the need for patients to prepare and administer the infusions themselves and potential site leakage during infusions. This device will administer therapy subcutaneously through single or multiple injection sites depending on the drug volumes prescribed.

While important in any medical setting, for home use, it is imperative that the infusion needle system assembly in particular be as simple and straightforward to use as possible.

To assist with ensuring that the needle reaches the desired tissues or structures, infusion needles are often provided in specific lengths. In addition, to help anchor the needle during the infusion procedure, these needles often have an anchor of sorts, from which the needle extends, and which is taped in place upon a patient once the needle element has been disposed into the tissue.

To further minimize the possibility of disturbing the needle, the medicant supply tubing is essentially parallel to the patient's skin, and capable of being taped in place while the delivery end of the needle is perpendicular to the skin and at the desired pre-determined depth. In some cases, such as in the case of butterfly needles, the butterfly needle itself may have an adhesive layer predisposed or otherwise incorporated in or upon the surface that is intended to contact the patient's skin.

Of course, to receive an infusion therapy, the patient or his or her caregiver must insert one or more needles into the patient's body. While some patients have no difficulty self-inserting needles or receiving needles in their body, other patients are sensitive to the pain of the injection or are uncomfortable seeing needles or injecting needles into their body. In particular, many children have difficulty receiving infusion therapy due to the pain of needle insertion or fear of needles.

The method in which the needle is injected into the tissue is relevant to preventing pain. Research has shown that the speed of the insertion of the needle is important to protect the tissue layers from excessive damage and reduce the pain of the insertion. When a needle is slowly inserted into the tissue, there is a more potential for damage to be observed in the tissue as compared to the rapid insertion of a needle into tissue.

Many of the therapies that require infusions are also given on a weekly or biweekly basis, and some even more frequently. As such, there is a desire among patients and caregivers for a simple and consistent system that provides consistent needle insertion, and potentially, a reduction in pain.

Needle insertion devices are known—indeed, US Patent Publication US 2009/0216215 to Thalmann sets forth a type of needle insertion device, which has a piston/thrust element 4 that is drawn back by hand to prepare the device for a needle insertion. For those with poor or limited hand strength, such an operation may be difficult. In addition, the Thalmann device is not appropriate for butterfly needles which have wings—the wings being advantageous for temporarily securing the inserted needle to the patient during the treatment session.

Another needle insertion device may be found in US Patent Publication US 2012/0245497 to Nicholls et al, but here again the device does not accommodate butterfly needles. U.S. Pat. No. 7,014,625 to Bengtsson discloses a Needle Insertion Device, wherein the needle and the insertion device are apparently inseparable and the entire insertion device is adhered to the patient during treatment and then removed—the device apparently being single use and therefore potentially costly.

Hence there is a need for a method and system that is capable of overcoming one or more of the above identified challenges.

### SUMMARY OF THE INVENTION

Our invention solves the problems of the prior art by providing a system and method for administering a butterfly needle to a patient.

In particular, and by way of example only, according to one embodiment of the present invention, provided is a system for administering a butterfly needle to a patient, including: a butterfly needle insertion device, comprising: a housing comprising a shell with a distal end and a proximal opening, the proximal opening having two opposing grooves each having a width to receive a wing of a butterfly needle, the opposing grooves extending from the proximal opening towards the distal end, and two opposing channels disposed between the opposing grooves, the opposing channels extending towards the distal end; a drive plunger occupying at least a portion of the space defined by the shell of the housing and in shifting engagement with the housing, wherein the drive plunger has a distal end to be engaged by a stop to hold the drive plunger in a retracted position and a proximal end structured and arranged to receive the butterfly needle with the wings extending through the opposing grooves and beyond the housing, a supply line of the butterfly needle received by one of the opposing channels; a butterfly needle retainer disposed within the housing and structured and arranged to temporarily hold the butterfly needle to the proximal end of the drive plunger when the drive plunger is in the retracted position prior to position prior to the butterfly needle insertion into the patient; a drive spring associated with the drive plunger such that when the drive plunger is driven into the housing to the retracted position, the drive spring is loaded and when the drive plunger is released, the drive plunger travels distally from the force of the drive spring unloading to deliver the butterfly needle into the patient.

For yet another embodiment, provided is a system for administering a butterfly needle to a patient, including: a butterfly needle insertion device, comprising: a housing comprising a shell with a distal end and a proximal opening, the proximal opening having two opposing grooves each having a width to receive a wing of a butterfly needle, the opposing grooves extending from the proximal opening towards the distal end, and two opposing channels disposed between the opposing grooves, the opposing channels extending towards the distal end; a drive plunger occupying at least a portion of the space defined by the shell of the housing and in shifting engagement with the housing, wherein the drive plunger has a distal end to be engaged by a stop to hold the drive plunger in a retracted position and a proximal end structured and arranged to receive the butterfly needle with the wings extending through the opposing grooves and beyond the housing, a supply line of the butterfly needle received by one of the opposing channels; a catch assembly disposed within the housing and providing an aperture through which at least a portion of the drive plunger moves, the catch assembly providing a pair of opposing catch springs disposed proximate to opposing sides of the drive plunger, the catch springs structured and arranged to temporarily hold the butterfly needle to the proximal end of the drive plunger when the drive plunger is in the retracted position prior to insertion of the butterfly needle into the patient; a drive spring associated with the drive plunger such that when the drive plunger is driven into the housing to the retracted position, the drive spring is loaded and when the drive plunger is released, the drive plunger travels distally from the force of the drive spring unloading to deliver the butterfly needle into the patient; and an inner shell disposed within the housing and about the drive plunger, the catch assembly and the drive spring, the inner shell having a proximal end providing a proximal surface disposed at least partially about the proximal opening.

In yet another embodiment, provided is a system for administering a butterfly needle to a patient, including: a butterfly needle insertion device, comprising: a housing comprising a shell with a distal end and a proximal opening, the proximal opening having two opposing grooves each having a width to receive a wing of a butterfly needle, the opposing grooves extending from the proximal opening towards the distal end, and two opposing channels disposed between the opposing grooves, the opposing channels extending towards the distal end; a drive plunger occupying at least a portion of the space defined by the shell of the housing and in shifting engagement with the housing, wherein the drive plunger has a distal end to be engaged by a stop to hold the drive plunger in a retracted position and a proximal end structured and arranged to receive the butterfly needle with the wings extending through the opposing grooves and beyond the housing, a supply line of the butterfly needle received by one of the opposing channels; a butterfly needle retainer disposed within the housing and structured and arranged to temporarily hold the butterfly needle to the proximal end of the drive plunger when the drive plunger is in the retracted position prior to insertion of the butterfly needle into the patient; a drive spring associated with the drive plunger such that when the drive plunger is driven into the housing to the retracted position, the drive spring is loaded and when the drive plunger is released, the drive plunger travels distally from the force of the drive spring unloading to deliver the butterfly needle into the patient; and an inserter base, the inserter base structured and arranged to receive the butterfly needle, the housing disposed over the inserter base and depressed down about the inserter base to retract the drive plunger within the housing and engage the butterfly needle to the butterfly needle retainer.

And, still further, for yet another embodiment, provided is a method for administering a butterfly needle to a patient, including: providing a butterfly needle insertion device, comprising: a housing comprising a shell with a distal end and a proximal opening, the proximal opening having two opposing grooves each having a width to receive a wing of a butterfly needle, the opposing grooves extending from the proximal opening towards the distal end, and two opposing channels disposed between the opposing grooves, the opposing channels extending towards the distal end; a drive plunger occupying at least a portion of the space defined by the shell of the housing and in shifting engagement with the housing, wherein the drive plunger has a distal end to be engaged by a stop to hold the drive plunger in a retracted position and a proximal end structured and arranged to receive the butterfly needle with the wings extending through the opposing grooves and beyond the housing, a supply line of the butterfly needle received by one of the opposing channels; a butterfly needle retainer disposed within the housing and structured and arranged to temporarily hold the butterfly needle to the proximal end of the drive plunger when the drive plunger is in the retracted position prior to insertion of the butterfly needle into the patient; a drive spring associated with the drive plunger such that when the drive plunger is driven into the housing to the retracted position, the drive spring is loaded and when the drive plunger is released, the drive plunger travels distally from the force of the drive spring unloading to deliver the butterfly needle into the patient; providing an inserter base, the inserter base structured and arranged to receive the butterfly needle, the housing disposed over the inserter base and depressed down about the inserter base to retract the drive plunger within the housing and engage the butterfly needle to the butterfly needle retainer; disposing the butterfly needle upon the inserter base; disposing the housing over the inserter base; depressing the housing down upon the inserter base to retract the drive plunger into the housing and engage the butterfly needle temporarily with the butterfly needle retainer, disposing the proximal opening over an intended infusion site of the patient; and releasing the stop to dispose the butterfly needle into the patient.

### BRIEF DESCRIPTION OF THE DRAWINGS

FIG. 1A is a front, upper perspective view of a butterfly needle insertion device in accordance with at least one embodiment of the present invention;

FIG. 1B is a front, lower perspective view of a butterfly needle insertion device in accordance with at least one embodiment of the present invention;

FIG. 1C is a plain side view of a butterfly needle insertion device in accordance with at least one embodiment of the present invention;

FIG. 1D is a plain front view of a butterfly needle insertion device in accordance with at least one embodiment of the present invention;

FIG. 1E is a bottom plane view of a butterfly needle insertion device in accordance with at least one embodiment of the present invention;

FIG. 2 is an exploded view of a butterfly needle insertion device with inserter base in accordance with at least one embodiment of the present invention;

FIG. 3 is an enlarged perspective view of a drive plunger element from the butterfly needle insertion device in accordance with at least one embodiment of the present invention;

FIG. 4A is a side cut through view of a butterfly needle insertion device disposed above and apart from a butterfly needle disposed upon an inserter base in accordance with at least one embodiment of the present invention;

FIG. 4B is a side cut through view of a butterfly needle insertion device disposed upon a butterfly needle disposed upon an inserter base as an intermediate step in loading the butterfly needle to the butterfly needle insertion device in accordance with at least one embodiment of the present invention;

FIG. 4C is a side cut through view of a butterfly needle insertion device fully disposed upon a butterfly needle disposed upon an inserter base as the ending step in loading the butterfly needle to the butterfly needle insertion device in accordance with at least one embodiment of the present invention;

FIG. 4D is a side cut through view of a butterfly needle insertion device loaded with a butterfly needle in accordance with at least one embodiment of the present invention; and

FIG. 5 is a bottom plane view of a butterfly needle insertion device loaded with a butterfly needle in accordance with at least one embodiment of the present invention;

### DETAILED DESCRIPTION

Before proceeding with the detailed description, it is to be appreciated that the present teaching is by way of example only, not by limitation. The concepts herein are not limited to use or application with a specific system or method for providing a system or method for a butterfly needle assembly. Thus, although the instrumentalities described herein are for the convenience of explanation shown and described with respect to exemplary embodiments, it will be understood and appreciated that the principles herein may be applied equally in other types of injection or infusion needle assembly systems and methods.

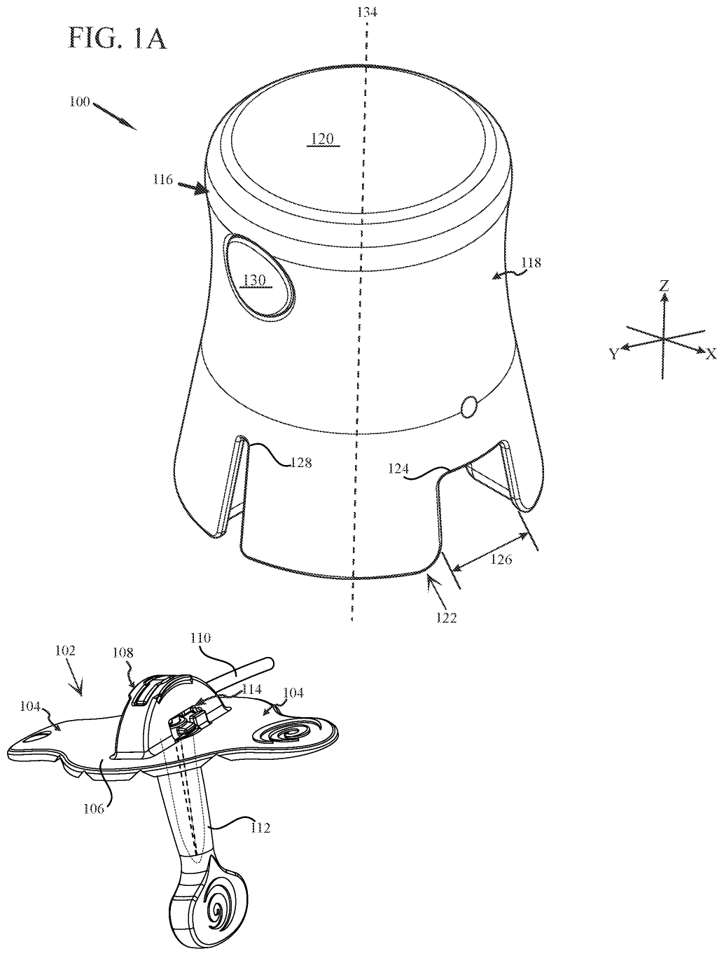

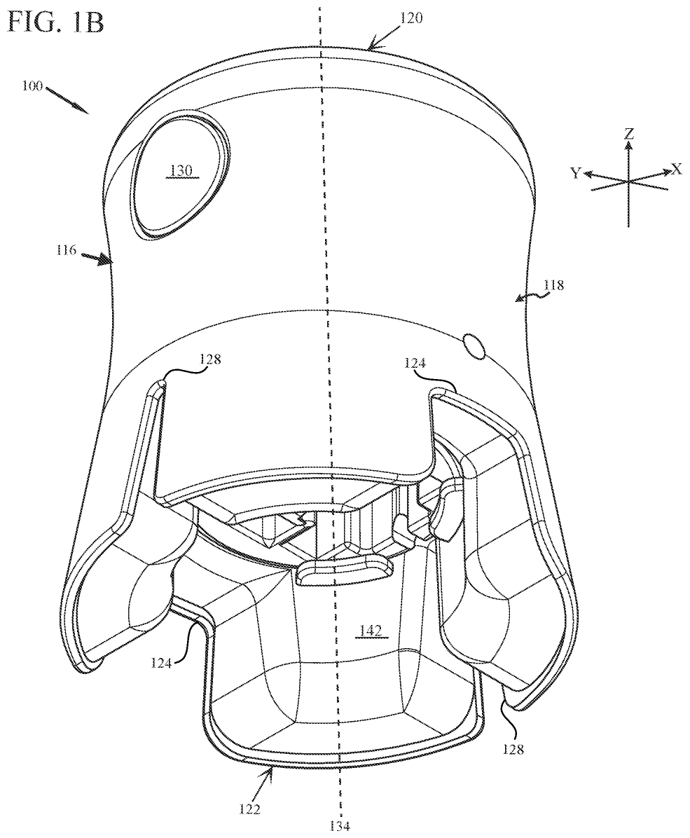

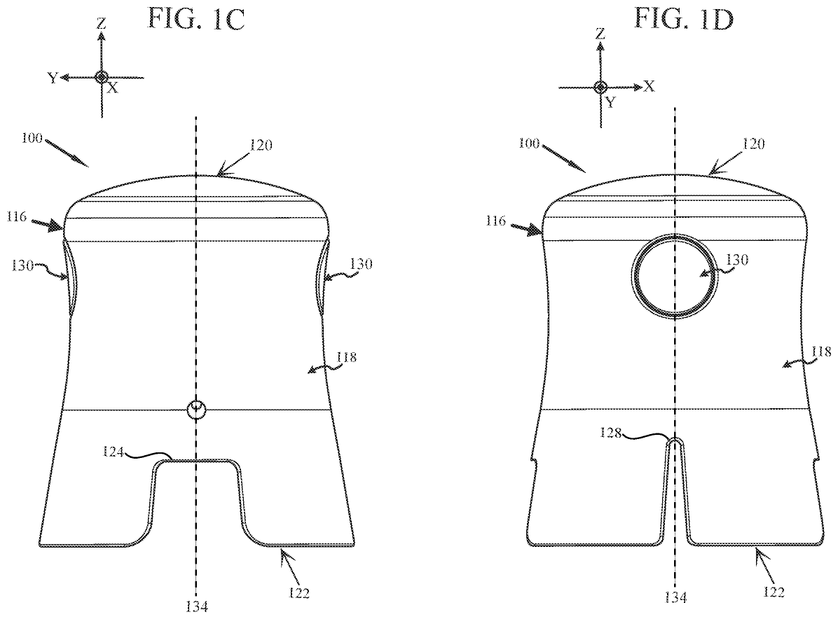

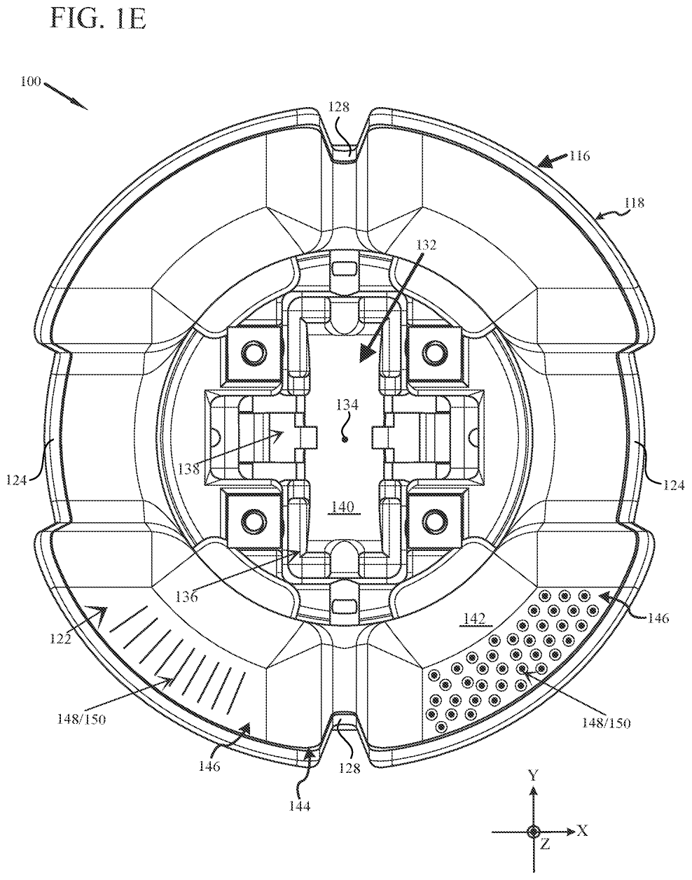

This invention is described with respect to preferred embodiments in the following description with reference to the Figures, in which like numbers represent the same or similar elements. Further, with the respect to the numbering of the same or similar elements, it will be appreciated that the leading values identify the Figure in which the element is first identified and described, e.g., element 100 appears in FIG. 1.

Turning now to the drawings, and more specifically FIGS. 1A-1E there is shown at least one embodiment of a butterfly needle insertion device 100, hereinafter BNID 100, which will be understood and appreciated as a key element of the system and method for administering a butterfly needle 102 to a patient.

FIG. 1A presents a top perspective view of BNID 100, with FIG. 1B providing a bottom perspective view, FIGS. 1C and 1D provide a plain side view and a plane front view, with FIG. 1E providing a plain bottom view.

To facilitate the description of systems and methods for embodiments of BNID 100, the orientation of BNID 100 as presented in the figures is referenced to the coordinate system with three axes orthogonal to one another as shown in FIG. 1. The axes intersect mutually at the origin of the coordinate system, which is chosen to be the center of BNID 100, however the axes shown in all figures are offset from their actual locations for clarity and ease of illustration.

As this continuing description will illuminate, BNID 100 is structured and arranged to advantageously facilitate the insertion of a butterfly needle 102 into a patient.

Although the BNID 100 may advantageously assist with the insertion of a variety of different butterfly needles 102, for at least one embodiment, BNID 100 has specific features that advantageously enhance the insertion of butterfly needles provided by KORU Medical Systems, Inc. of Mahwah, New Jersey. KORU Medical Systems, Inc. of Mahwah, New Jersey, is and has been a pioneer in needle set technology and provides butterfly needles as set forth in U.S. application Ser. No. 18/216,342 entitled SYSTEM AND METHOD FOR BUTTERFLY NEEDLE ASSEMBLY, incorporated herein by reference. Indeed, for purposes of ease of illustration and discussion, the exemplary butterfly needle 102 as shown in the drawings, will be understood and appreciated as an embodiment of a butterfly needle in accordance with U.S. application Ser. No. 18/216,342, now U.S. patent Ser. No. ______, incorporated herein by reference.

For ease of discussion, it will be understood and appreciated that butterfly needle 102 provides opposing wings 104 extending away from a central region 106, the central region 106 also providing a needle hub 108, with a supply line 110. It will also be understood and appreciated, that at least some butterfly needles 102 will be provided with a needle sheath 112 which protectively covers the needle prior to use to protect from accidental needle stick and maintain sterility of the needle. The needle hub 108 may also provide side apertures 114. Of course, it will be understood and appreciated, that various embodiments of BNID 100 may be adapted for the specific elements of other butterfly needles besides those of U.S. application Ser. No. 18/216,342—and even use with a plurality of different, e.g., generic, butterfly needles without departing from the teachings herein.

As shown, BNID 100 is generally provided by a housing 116 comprising a shell 118. For at least one embodiment this shell 118 may be understood and appreciated as an outer shell, as an inner shell will be discussed below. The shell 118 has a solid distal end 120 and a proximal opening 122 opposite from the distal end 120. The proximal opening 122 has two opposing grooves 124 each having a width 126 sized to receive a wing 104 of a butterfly needle 102. As may be appreciated most easily in FIGS. 1A and 1B, these opposing grooves 124 extend from the proximal opening 122 towards the distal end 120.

The shell 118 also has two opposing channels 128 disposed between the opposing grooves 124, these opposing channels 128 likewise extending from the proximal opening 122 towards the distal end 120. These opposing channels 128 have a width sized to receive supply tubing that couples the butterfly needle 102 to a fluid reservoir (not shown). It will be appreciated that for at least one embodiment the opposing grooves 124 are substantially perpendicular to the opposing channels 128.

With respect to both the opposing grooves 124 and the opposing channels 128, it will be understood and appreciated that each extends from the proximal opening 122 towards the distal end 120 for a distance that is, in general, at least equal to if not slightly longer than the length of the needle, or longest needle, for which an embodiment of BNID 100 is intended for use.

For at least one embodiment, the housing 116 also has opposing buttons 130 at least partially disposed within the side of the housing 116, and more specifically the shell 118, proximate to the distal end 120. For at least one embodiment, the buttons 130 extend outward from the housing such that they can be easily felt and recognized by a user through tactile sensation. For yet another embodiment, the buttons 130 may be essentially flush with the shell 118. Although shown as round buttons, it will be understood and appreciated that in varying embodiments they may be various other geometric shapes, and for at least one embodiment may even appear as a compressible band around the shell 118 without departure from the scope of invention.

As may be appreciated in FIG. 1E, the plain bottom view, for at least one embodiment, BNID 100 includes a drive plunger 132 within the shell 118. As will be further discussed below, the plunger is in shifting engagement with the housing, transitioning from an extended or deployed state to a retracted state generally by lateral movement within the housing 116 along the longitudinal centerline 134 of the housing 116.

The drive plunger 132 has a proximal end 136 that is structured and arranged to receive the butterfly needle 102, and more specifically—for at least one embodiment, the needle hub of the butterfly needle 102. The drive plunger 132 also has a distal end (not shown in FIGS. 1A-1E) to be engaged by a stop to hold the drive plunger 132 in a retracted position.

As may also be appreciated in FIG. 1E, BNID 100 also provides a butterfly needle retainer 138 disposed within the housing and structured and arranged to temporarily hold the butterfly needle 102 to the proximal end 136 of the drive plunger 132 when the drive plunger 132 is in a retracted position prior to insertion of the butterfly needle 102 into the patient.

As is conceptually shown in the drawings, the needle hub 108 has a rounded profile. Accordingly, for at least one embodiment, the proximal end 136 of the drive plunger 132 has a concave seat 140 structured and arranged to receive the needle hub 108 of the butterfly needle 102.

As noted above, shell 118 may in some embodiments be appreciated as an outer shell. Although for at least one embodiment, BNID 100 may indeed have one shell that is fabricated to a desired thickness and purpose, for at least one alternative embodiment the fabrication and assembly may be simplified by providing an inner shell 142. More specifically, as may be appreciated in FIGS. 1B and 1E, for at least one embodiment, BNID 100 further includes an inner shell 142 disposed about the drive plunger 132 and the butterfly needle retainer 138.

The inner shell 142 has a proximal end 144 disposed at least partially about the proximal opening 122. For at least one embodiment, this proximal end 144 may provide a proximal surface 146, which may optionally provide a component 148 such as ridges, points, bumps or other texture 150 for relieving discomfort associated with injecting the butterfly needle 102 into the patient receiving the infusion. Moreover, a plurality of contact points has been found to provide additional nerve stimulus that may mask, or at least desensitize the localized prick of the needle insertion. For at least one embodiment, the component 148 may also include a vibrator component. Further, in at least one embodiment such a vibrator element is provided a vibrator motor and power source (not shown) which may be activated by a pressure switch when the BNID 100 is disposed upon the patient, or activation of the buttons 130, or activation of a separate vibration button. For yet another embodiment, the vibrator element may be provided by a mechanical system including an off-center weight and spring system that is associated with the drive plunger 132 further described below.

With respect to FIG. 1E, the plain bottom view, it will be understood and appreciated that BNID 100 is a substantially symmetrical device. As such it may receive a butterfly needle 102 without great care to assure that it is aligned in one specific orientation. Moreover, it will be understood and appreciated that BNID 100 can accommodate a butterfly needle 102 in either a forward or backward orientation—the wings extend through the opposing grooves 124, and the supply tubing will extend through one opposing channel 128 or the other.

With respect to FIGS. 1A-1E, it will be understood and appreciated that at least one embodiment of BNID 100 provides the visual appearance of a simple and uncomplicated device.

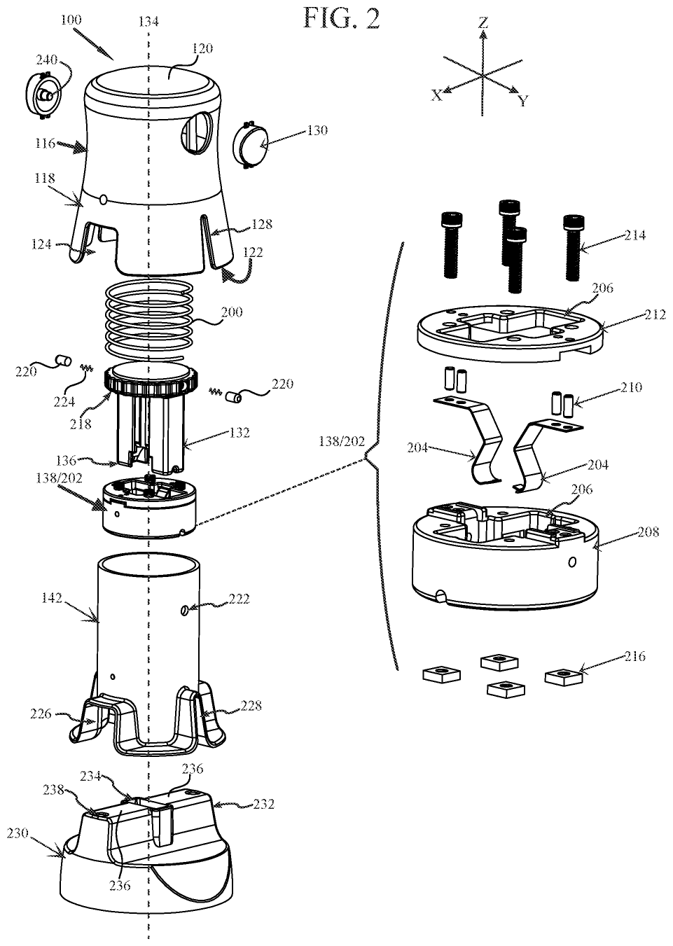

FIG. 2 is an exploded view of an embodiment of BNID 100 so as to more fully appreciate the internal elements. More specifically, as shown in FIG. 2, for at least one embodiment, BNID 100 has a housing 116 comprising a shell 118, which as discussed above has a distal end 120 and a proximal opening 122 opposite from the distal end 120.

Within the shell 118 are a drive plunger 132, a drive spring 200, a butterfly needle retainer 138, and an inner shell 142 which is disposed within the shell 118 and about the drive plunger 132, the drive spring 200, and the butterfly needle retainer 138.

As may be more fully appreciated in the enlarged and exploded depiction of the butterfly needle retainer 138, for at least one embodiment the butterfly needle retainer 138 may be further described as a catch assembly 202 providing opposing catch springs 204 which are structured and arranged to temporarily engaged and retain the butterfly needle 102, and more specifically the needle hub 108 of the butterfly needle 102. For a butterfly needle 102 providing side apertures 114 in the needle hub 108, the catch springs 204 may be positioned strategically so as to temporarily engage with these side apertures 114 for an improved temporary hold upon the retained butterfly needle 102.

More specifically, the catch assembly 202 provides an aperture 206 through which at least a portion of the drive plunger 132 moves. The catch assembly may be provided by a catch base 208 that receives the catch springs 204 on opposing sides of the aperture 206, the catch springs 204 affixed in place by mounting pins 210. For at least one embodiment, a catch top 212 is affixed to the catch base 208, such as by bolts 214 and nuts 216. Of course, those skilled in the art will understand and appreciate that the catch springs 204 may be affixed in place by a structure other than mounting pins, and the catch top 212 may be affixed to the catch base with structures other than nuts and bolts. Moreover, variations in embodiments of the catch assembly 202 providing the catch springs 204 beyond the specific example recited may be substituted without departure from the scope of the present invention.

The catch assembly 202 is disposed and affixed within the housing slightly below the upper end of the opposing grooves 124. The distal end 218 of the drive plunger 132 may now be appreciated in FIG. 2, as well as button catches 220 which are in sliding arrangement through holes 222 in inner shell 142 with buttons 130. Compression springs 224 drive button catches 220 into through holes 222 and thereby stop and retain the drive plunger. When buttons 130 are depressed, inner pins 240 depress button catches 220 back through holes 222 and thereby release the drive plunger 132. Of course, those skilled in the art will understand and appreciate that other elements and systems may be used to temporarily retain the drive plunger 132 in a retracted state without departure from the scope of the present invention.

For at least one embodiment a compression drive spring is disposed between the top of the distal end 218 of the drive plunger 132 and the underside of the distal end 120 of the housing 116. Alternatively, an optional embodiment of BNID 100 is provided by an embodiment incorporating a tension drive spring 200 that is disposed about the drive plunger and between the catch assembly 202 and the distal end 218 of the drive plunger (the spring 200 is not shown in this position). For yet a third embodiment, two drive springs 200—both a tension spring and a compression spring—may be used, the compression drive spring above the drive plunger 132 and the tension drive spring disposed about the lower portion of the drive plunger 132.

More specifically, both a compression spring and a tension spring have an unloaded state when at rest in their initial, uncompressed or un-stretched state. The action of transitioning a compression spring from an uncompressed to a compressed state, loads the compression spring with force, and likewise the action of transitioning the tension spring from an unstretched state to stretched state loads the tension spring with force. When each spring is released, e.g., unloaded, so too is the force, which is used to drive the drive plunger from its retracted position to a deployed position. Of course, it will be understood and appreciated that a compression drive spring may simply be disposed between the top of the distal end 218 of the drive plunger and the underside of the distal end 120 of the housing 116, whereas a tension drive spring 200 is hooked or otherwise affixed between the catch assembly 202 and the distal end 218 of the drive plunger 132.

As may also be appreciated in FIG. 2, the inner shell 142 has corresponding opposing grooves 226 and opposing channels 228 that are structured and arranged to align with the opposing grooves 124 and opposing channels 128 of the shell 118.

Also shown in FIG. 2 is an inserter base 230. The inserter base 230 has a raised midsection 232 that is structured and arranged to receive the butterfly needle 102. More specifically, the inserter base 230 has a recess 234 structured and arranged to receive the needle element of the butterfly needle 102. Advantageously, for at least one embodiment this recess 234 is sized to accommodate a sheath 112 disposed about the actual needle element.

Inserter base 230 also provides supports 236 to either side of the recess 234 upon which the butterfly needle is rested. As for at least one embodiment, the butterfly needle 102 may provide a closure system to close about the needle after use—such as a button and hole system disposed proximate to the edges of each wing; the inserter base 230 provides sockets 238 to receive a button if provided by a wing.

For at least one embodiment, the inserter base 230 is incorporated as a component of a disposable tray or packaging that provides the butterfly needle 102. As such a tray with the butterfly needle 102 is pre-assembled, sterilized and hermetically sealed by the providing facility, such a butterfly needle may not have a needle sheath 112 as the inserter base 230 itself upon which the butterfly needle has been disposed acts as the needle sheath. When BNID 100 is disposed upon the inserter base 230 component of such a tray and the butterfly needle 102 subsequently engaged to BNID 100, it will be understood and appreciated that the needle will be safely retained within the lower portion of BNID 100. The absence of an addition sheath, advantageously saves on the fabrication of sheath as an additional element, the time and cost of its installation about the needle, its subsequent disposal, and the need for the user to remove the sheath which also removes an opportunity for possible accidental needle stick.

As has been noted above with respect to BNID 100 in general, the inserter base 230 also will be appreciated to be substantially symmetrical, and as such may receive a butterfly needle 102 without specific orientation of only one position. More specifically, BNID 100 is advantageously bi-directional—able to receive a butterfly needle 102 in either of two orientation—the supply line 110 oriented towards the front or towards the back. In either configuration, the wings 104 will be disposed through the opposing grooves 124 and the supply line 110 will be disposed in either of the two opposing channels 128.

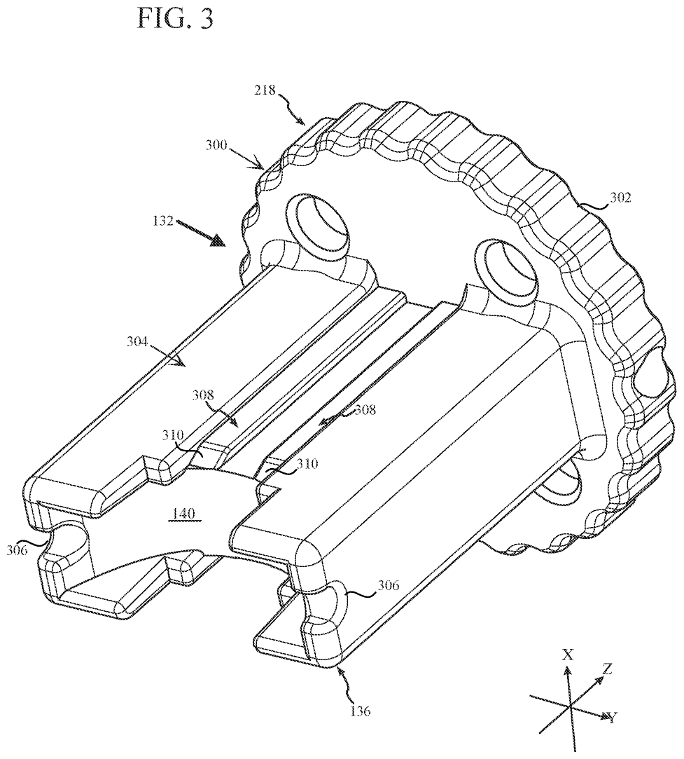

FIG. 3 provides an enlarged side perspective view of the drive plunger 132 in accordance with at least one embodiment. More specifically, for at least one embodiment the drive plunger 132 is appreciated to have a distal end 218 which is configured as a generally round element 300. This round element may have a plurality of side grooves 302 that are structured and arranged to mate with corresponding longitudinal ridges on the inside of the inner shell, and which collectively interact to ensure that during lateral translation of the drive plunger 132 within the housing, rotation of the drive plunger 132 is prevented.

The drive plunger 132 also consists of a shaft 304, which for at least one embodiment is a generally rectangular element extending perpendicularly from the distal end 120. It will also be appreciated that the cross section of the shaft 304 is structured and arranged for smooth passage through the aperture 206 in the catch assembly 202. And, as the cross section of the shaft 304 and aperture 206 are for at least one embodiment, not round, they cooperatively interact to prevent rotation of the drive plunger during lateral translation within the housing 116.

For the exemplary embodiment shown, the shaft 304 provides the proximal end 136 of the drive plunger 132. As may be further appreciated in FIG. 3, for at least one embodiment the proximal end 136 provides a concave seat 140. It will also be appreciated that on either end of the concave seat 140 is a notch 306. Depending on which way the butterfly needle 102 is positioned, as the concave seat 140 mates upon the needle hub, one notch 306 or the other will be disposed over and upon the supply line extending from the butterfly needle 102. Again, as there are opposing notches 306, the butterfly needle may be disposed upon the inserter base either forwards or backwards.

It will also be appreciated in FIG. 3 that the shaft 304 of the drive plunger has cam surfaces 308. These cam surfaces 308 are structured and arranged to engage with the catch springs 204 of the catch assembly 202 and drive them apart so as to release the butterfly needle as the drive plunger 132 moves to expel the butterfly needle from the BNID 100 and into the patient. Indeed, each cam surface 308 has as tapered end 310 so as to smoothly engage with the catch springs 204, thereby smoothly transitioning the catch springs 204 from an open to closed position and vice versa.

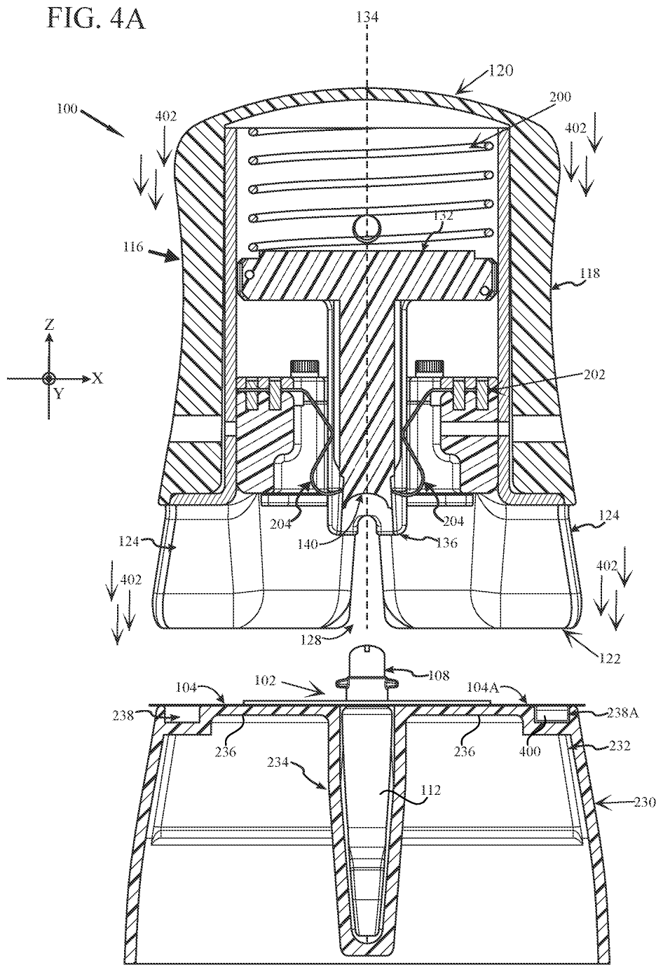

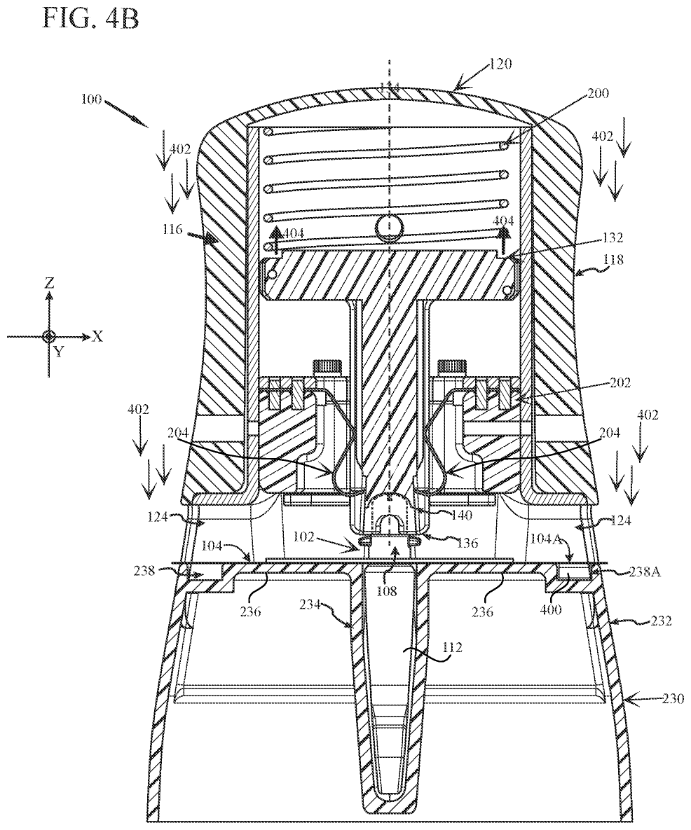

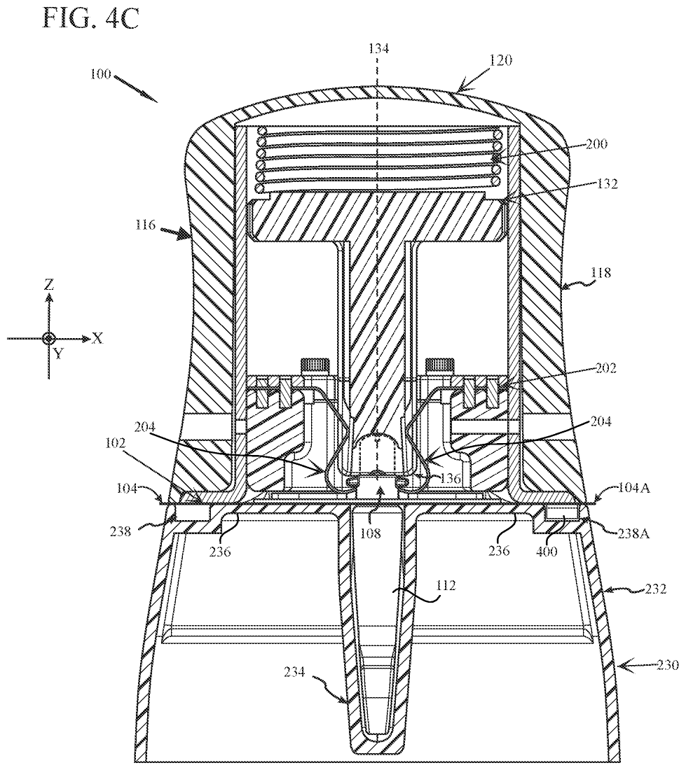

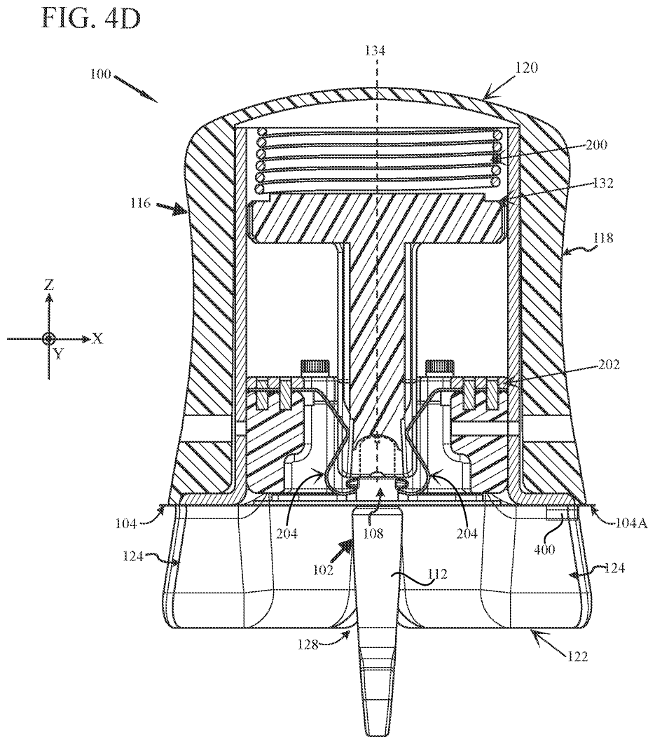

Having generally described the BNID 100 and inserter base 230, FIGS. 4A-4D provide side cut through views to illustrate their use for the loading of a butterfly needle 102 into BNID 100 for subsequent insertion into a patient. Moreover, it will be visually apparent that the present invention for a system for administering a butterfly needle to a patient does not require great dexterity, does not require the user to use his or her fingers to press the needle up into the housing 116, and permits the actual needle element of the butterfly needle to be protected and sterile until it is actually time for insertion into the patient.

It will be understood and appreciated that for ease and simplicity of use, the inserter base 230 is to be disposed by the user upon a flat and stable surface, such as a table, counter or other appropriate and available surface.

FIG. 4A presents an initial view of the butterfly needle 102 loading process. More specifically, a butterfly needle 102 is shown disposed upon the inserter base 230. The sheath 112 about the actual needle element is appreciated to be disposed into the recess 234 of the inserter base 230. With the wings 104 laid atop the supports 236 of the raised midsection 232. A closing button 400 of one wing 104A is also shown disposed in socket 238A.

As may be appreciated from the cut through view of BNID 100, the drive plunger 132 is in the initial position, with the proximal end 136 extending well below the catch base 208. It will also be appreciated that the drive spring 200 is also in an initial position, which is to say at rest and unloaded, as it has not been stretched into a tensioned position. The catch springs 204 are also at rest on either side of the shaft 304 of the drive plunger 132. As indicated by motion arrows 402, BIND 100 is being directed down and upon the inserter base 230.

In FIG. 4B, the position of BNID 100 has progressed such that it is now disposed over the inserter base 230. The needle hub 108 of the butterfly needle is received by the concave seat 140 of the drive plunger 132. As the downward motion of the housing 116 continues indicated by motion arrows 402, the butterfly needle 102 in contact with the proximal end 136, and more specifically the concave seat 140, of the drive plunger 132 laterally transitions the drive plunger 132 up and into the housing 116 along the longitudinal centerline 134 as shown by arrows 404—eventually to a recessed position as shown in FIG. 4C.

In FIG. 4C, the BNID 100 is fully disposed upon the inserter base 230. The drive plunger 132 has reached its retracted position, and is now held by the stop provided in at least one embodiment by the button catches 220 driven by compression springs 224 to engage with the holes 222 in inner shell 142, the button catches to be depressed and release the drive plunger 132 by depressing the buttons 130 (not shown in this view, see FIG. 2). Moreover, the drive spring 200, as a tension spring, has been stretched such that it is now loaded with a retractive force, which when released will pull the drive plunger 132 down and back to its protruding position—which in turn will quickly and efficiently insert the butterfly needle into the patient.

As may also be appreciated in FIG. 4C, the catch springs 204 of the catch assembly 202 have now engaged with the sides of the needle hub 108, and as such, the butterfly needle 102 is temporarily retained by the BNID 100 against the concave seat 140 of the drive plunger 132.

FIG. 4D provides a cross section view of BNID 100 now removed from the inserter base 230, and essentially ready for use, save of course for the removal of the sheath 112, if present. It will also be understood and appreciated, that with the butterfly needle 102 now engaged to BNID 100, the actual needle element of butterfly needle 102 is fully disposed within the housing 116. When the sheath 112 is removed, if present, BNID 100 with the butterfly needle 102 engaged does not present a significant needle stick risk to the patient or BNID 100 operator.

Again, it will be understood and appreciated that the catch assembly 202, and more specifically the catch springs 204 are temporarily retaining the butterfly needle 102 firmly against the concave seat 140 of the drive plunger 132. As noted, the drive spring 200 is now loaded with retained force, and upon release of the stop—e.g., by depression of the buttons 130 (not shown in FIGS. 4A-4D), the spring will retract to its initial state and the butterfly needle 102 will be driven down and into the patient.

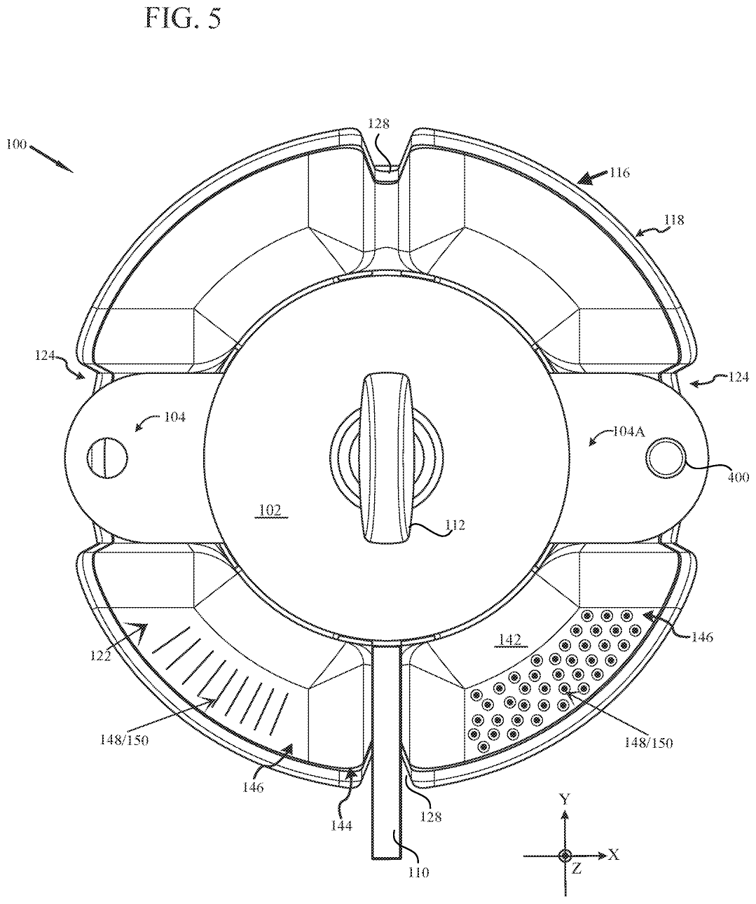

FIG. 5 provides a plain bottom view of BNID 100 similar to the plain bottom view of FIG. 1E described above, now with the butterfly needle 102 engaged. With respect to FIGS. 4C, 4D and 5, it will be appreciated that the wings 104 of the butterfly needle 102 do extend beyond the housing 104. It will be understood and appreciated that as the wing tips may be observed from above or the side of BNID 100, a user need not invert the BNID 100 to visually confirm the presence or absence of a butterfly needle 102.

To summarize the above descriptions, for at least one embodiment, the present invention provides a system for administering a butterfly needle 102 to a patient, comprising: a butterfly needle insertion device 100, comprising: a housing 116 comprising a shell 118 with a distal end 120 and a proximal opening 122, the proximal opening 122 having two opposing grooves 124 each having a width to receive a wing 104 of a butterfly needle 102, the opposing grooves 124 extending from the proximal opening 122 towards the distal end 120, and two opposing channels 128 disposed between the opposing grooves 124, the opposing channels 128 extending towards the distal end 120; a drive plunger 132 occupying at least a portion of the space defined by the shell 118 of the housing 116 and in shifting engagement with the housing 116, wherein the drive plunger 132 has a distal end 120 to be engaged by a stop to hold the drive plunger 132 in a retracted position and a proximal end 136 structured and arranged to receive the butterfly needle 102 with the wings 104 extending through the opposing grooves 124 and beyond the housing 116, a supply line of the butterfly needle 102 received by one of the opposing channels 128; a butterfly needle 102 retainer disposed within the housing 116 and structured and arranged to temporarily hold the butterfly needle 102 to the proximal end 136 of the drive plunger 132 when the drive plunger 132 is in the retracted position prior to insertion of the butterfly needle 102 into the patient; a drive spring 200 associated with the drive plunger 132 such that when the drive plunger 132 is driven into the housing 116 to the retracted position, the drive spring 200 is loaded and when the drive plunger 132 is released, the drive plunger 132 travels distally from the force of the drive spring 200 unloading to deliver the butterfly needle 102 into the patient.

For yet another embodiment, the present invention may be summarized as a system for administering a butterfly needle 102 to a patient, comprising: a butterfly needle insertion device 100, comprising: a housing 116 comprising a shell 118 with a distal end 120 and a proximal opening 122, the proximal opening 122 having two opposing grooves 124 each having a width to receive a wing 104 of a butterfly needle 102, the opposing grooves 124 extending from the proximal opening 122 towards the distal end 120, and two opposing channels 128 disposed between the opposing grooves 124, the opposing channels 128 extending towards the distal end 120; a drive plunger 132 occupying at least a portion of the space defined by the shell 118 of the housing 116 and in shifting engagement with the housing 116, wherein the drive plunger 132 has a distal end 120 to be engaged by a stop to hold the drive plunger 132 in a retracted position and a proximal end 136 structured and arranged to receive the butterfly needle 102 with the wings 104 extending through the opposing grooves 124 and beyond the housing 116, a supply line of the butterfly needle 102 received by one of the opposing channels 128; a butterfly needle 102 retainer disposed within the housing 116 and structured and arranged to temporarily hold the butterfly needle 102 to the proximal end 136 of the drive plunger 132 when the drive plunger 132 is in the retracted position prior to insertion of the butterfly needle 102 into the patient; a drive spring 200 associated with the drive plunger 132 such that when the drive plunger 132 is driven into the housing 116 to the retracted position, the drive spring 200 is loaded and when the drive plunger 132 is released, the drive plunger 132 travels distally from the force of the drive spring 200 unloading to deliver the butterfly needle 102 into the patient; and an inserter base 230, the inserter base 230 structured and arranged to receive the butterfly needle 102, the housing 116 disposed over the inserter base 230 and depressed down about the inserter base 230 to retract the drive plunger 132 within the housing 116 and engage the butterfly needle 102 to the butterfly needle 102 retainer.

It will also be understood and appreciated that at least one embodiment of the present invention may be that of a method for administering a butterfly needle to a patient. Such a method may be summarized as providing a butterfly needle insertion device 100, comprising: a housing 116 comprising a shell 118 with a distal end 120 and a proximal opening 122, the proximal opening 122 having two opposing grooves 124 each having a width to receive a wing 104 of a butterfly needle 102, the opposing grooves 124 extending from the proximal opening 122 towards the distal end 120, and two opposing channels 128 disposed between the opposing grooves 124, the opposing channels 128 extending towards the distal end 120; a drive plunger 132 occupying at least a portion of the space defined by the shell 118 of the housing 116 and in shifting engagement with the housing 116, wherein the drive plunger 132 has a distal end 120 to be engaged by a stop to hold the drive plunger 132 in a retracted position and a proximal end 136 structured and arranged to receive the butterfly needle 102 with the wings 104 extending through the opposing grooves 124 and beyond the housing 116, a supply line of the butterfly needle 102 received by one of the opposing channels 128; a butterfly needle 102 retainer disposed within the housing 116 and structured and arranged to temporarily hold the butterfly needle 102 to the proximal end 136 of the drive plunger 132 when the drive plunger 132 is in the retracted position prior to insertion of the butterfly needle 102 into the patient; a drive spring 200 associated with the drive plunger 132 such that when the drive plunger 132 is driven into the housing 116 to the retracted position, the drive spring 200 is loaded and when the drive plunger 132 is released, the drive plunger 132 travels distally from the force of the drive spring 200 unloading to deliver the butterfly needle 102 into the patient; providing an inserter base 230, the inserter base 230 structured and arranged to receive the butterfly needle 102, the housing 116 disposed over the inserter base 230 and depressed down about the inserter base 230 to retract the drive plunger 132 within the housing 116 and engage the butterfly needle 102 to the butterfly needle 102 retainer; disposing the butterfly needle 102 upon the inserter base 230; disposing the housing 116 over the inserter base 230; depressing the housing 116 down upon the inserter base 230 to retract the drive plunger 132 into the housing 116 and engage the butterfly needle 102 temporarily with the butterfly needle 102 retainer; disposing the proximal opening 122 over an intended infusion site of the patient; and releasing the stop to dispose the butterfly needle 102 into the patient.

Changes may be made in the above methods, systems and structures without departing from the scope hereof. It should thus be noted that the matter contained in the above description and/or shown in the accompanying drawings should be interpreted as illustrative and not in a limiting sense. Indeed, many other embodiments are feasible and possible, as will be evident to one of ordinary skill in the art. The claims that follow are not limited by or to the embodiments discussed herein, but are limited solely by their terms and the Doctrine of Equivalents.

What is claimed:

1. A system for administering a butterfly needle to a patient, comprising:
a butterfly needle insertion device, comprising:
a housing comprising a shell with a distal end and a proximal opening, the proximal opening having two opposing grooves each having a width to receive a wing of a butterfly needle, the opposing grooves extending from the proximal opening towards the distal end, and two opposing channels disposed between the opposing grooves, the opposing channels extending towards the distal end;
a drive plunger occupying at least a portion of the space defined by the shell of the housing and in shifting engagement with the housing, wherein the drive plunger has a distal end to be engaged by a stop to hold the drive plunger in a retracted position and a proximal end structured and arranged to receive the butterfly needle with the wings extending through the opposing grooves and beyond the housing, a supply line of the butterfly needle received by one of the opposing channels;
a butterfly needle retainer disposed within the housing and structured and arranged to temporarily hold the butterfly needle to the proximal end of the drive plunger when the drive plunger is in the retracted position prior to insertion of the butterfly needle into the patient;
a drive spring associated with the drive plunger such that when the drive plunger is driven into the housing to the retracted position, the drive spring is loaded and when the drive plunger is released, the drive plunger travels distally from the force of the drive spring unloading to deliver the butterfly needle into the patient.

2. The system of claim 1, further including an inner shell disposed within the housing and about the drive plunger, the butterfly needle retainer and the drive spring, the inner shell having a proximal end providing a proximal surface disposed at least partially about the proximal opening.

3. The system of claim 2, further including a component for relieving discomfort associated with injecting the butterfly needle into the patient receiving the injection.

4. The system of claim 3, wherein the component for relieving discomfort further includes a textured pad on at least a portion of the proximal surface.

5. The system of claim 3, wherein the component for relieving discomfort further includes a vibrator component.

6. The system of claim 1, further including an inserter base, the inserter base structured and arranged to receive the butterfly needle, the housing disposed over the inserter base and depressed down about the inserter base to retract the drive plunger within the housing and engage the butterfly needle to the butterfly needle retainer.

7. The system of claim 6, wherein the inserter base is further structured and arranged to receive the butterfly needle with a needle sheath in place.

8. The system of claim 6, wherein the inserter base is provided as a component of a tray providing the butterfly needle, the inserter base of the tray further maintaining a sterile condition of the butterfly needle at least until the butterfly needle is received by the butterfly needle insertion device.

9. The system of claim 1, wherein the butterfly needle retainer is a catch assembly disposed within the housing and providing an aperture through which at least a portion of the drive plunger moves, the catch assembly providing a pair of opposing catch springs disposed proximate to opposing sides of the drive plunger.

10. The system of claim 1, wherein the stop is provided by opposing buttons extending from sides of the housing, depression of the buttons releasing the drive plunger from its retracted state.

11. The system of claim 1, wherein the proximal end of the drive plunger has a concave seat structured and arranged to receive the needle hub of the butterfly needle.

12. The system of claim 1, wherein the opposing grooves are substantially perpendicular to the opposing channels.

13. A system for administering a butterfly needle to a patient, comprising:
a butterfly needle insertion device, comprising:
a housing comprising a shell with a distal end and a proximal opening, the proximal opening having two opposing grooves each having a width to receive a wing of a butterfly needle, the opposing grooves extending from the proximal opening towards the distal end, and two opposing channels disposed between the opposing grooves, the opposing channels extending towards the distal end;
a drive plunger occupying at least a portion of the space defined by the shell of the housing and in shifting engagement with the housing, wherein the drive plunger has a distal end to be engaged by a stop to hold the drive plunger in a retracted position and a proximal end structured and arranged to receive the butterfly needle with the wings extending through the opposing grooves and beyond the housing, a supply line of the butterfly needle received by one of the opposing channels;
a catch assembly disposed within the housing and providing an aperture through which at least a portion of the drive plunger moves, the catch assembly providing a pair of opposing catch springs disposed proximate to opposing sides of the drive plunger, the catch springs structured and arranged to temporarily hold the butterfly needle to the proximal end of the drive plunger when the drive plunger is in the retracted position prior to insertion of the butterfly needle into the patient;
a drive spring associated with the drive plunger such that when the drive plunger is driven into the housing to the retracted position, the drive spring is loaded and when the drive plunger is released, the drive plunger travels distally from the force of the drive spring unloading to deliver the butterfly needle into the patient; and
an inner shell disposed within the housing and about the drive plunger, the catch assembly and the drive spring, the inner shell having a proximal end providing a proximal surface disposed at least partially about the proximal opening.

14. The system of claim 13, further including an inserter base, the inserter base structured and arranged to receive the butterfly needle, the housing disposed over the inserter base and depressed down about the inserter base to retract the drive plunger within the housing and engage the butterfly needle to the butterfly needle retainer.

15. The system of claim 14, wherein the inserter base is further structured and arranged to receive the butterfly needle with a needle sheath in place.

16. The system of claim 14, wherein the inserter base is provided as a component of a tray providing the butterfly needle, the inserter base of the tray further maintaining a sterile condition of the butterfly needle at least until the butterfly needle is received by the butterfly needle insertion device.

17. The system of claim 13, further including a component for relieving discomfort associated with injecting the butterfly needle into the patient receiving the injection.

18. The system of claim 17, wherein the component for relieving discomfort further includes a textured pad on at least a portion of the distal surface.

19. The system of claim 17, wherein the component for relieving discomfort further includes a vibrator component.

20. The system of claim 13, wherein the stop is provided by opposing buttons extending from sides of the housing, depression of the buttons releasing the drive plunger from its retracted state.

21. The system of claim 13, wherein the proximal end of the drive plunger has a concave seat structured and arranged to receive the needle hub of the butterfly needle.

22. The system of claim 13, wherein the opposing grooves are substantially perpendicular to the opposing channels.

23. A system for administering a butterfly needle to a patient, comprising:
a butterfly needle insertion device, comprising:
a housing comprising a shell with a distal end and a proximal opening, the proximal opening having two opposing grooves each having a width to receive a wing of a butterfly needle, the opposing grooves extending from the proximal opening towards the distal end, and two opposing channels disposed between the opposing grooves, the opposing channels extending towards the distal end;
a drive plunger occupying at least a portion of the space defined by the shell of the housing and in shifting engagement with the housing, wherein the drive plunger has a distal end to be engaged by a stop to hold the drive plunger in a retracted position and a proximal end structured and arranged to receive the butterfly needle with the wings extending through the opposing grooves and beyond the housing, a supply line of the butterfly needle received by one of the opposing channels;
a butterfly needle retainer disposed within the housing and structured and arranged to temporarily hold the butterfly needle to the proximal end of the drive plunger when the drive plunger is in the retracted position prior to insertion of the butterfly needle into the patient;
a drive spring associated with the drive plunger such that when the drive plunger is driven into the housing to the retracted position, the drive spring is loaded and when the drive plunger is released, the drive plunger travels distally from the force of the drive spring unloading to deliver the butterfly needle into the patient; and
an inserter base, the inserter base structured and arranged to receive the butterfly needle, the housing disposed over the inserter base and depressed down about the inserter base to retract the drive plunger within the housing and engage the butterfly needle to the butterfly needle retainer.

24. The system of claim 23, further including an inner shell disposed within the housing and about the drive plunger, the butterfly needle retainer and the drive spring, the inner shell having a proximal end providing a proximal surface disposed at least partially about the proximal opening.

25. The system of claim 24, further including a component for relieving discomfort associated with injecting the butterfly needle into the patient receiving the injection.

26. The system of claim 25, wherein the component for relieving discomfort further includes a textured pad on at least a portion of the distal surface.

27. The system of claim 25, wherein the component for relieving discomfort further includes a vibrator component.

28. The system of claim 23, wherein the inserter base is further structured and arranged to receive the butterfly needle with a needle sheath in place.

29. The system of claim 23, wherein the inserter base is provided as a component of a tray providing the butterfly needle, the inserter base of the tray further maintaining a sterile condition of the butterfly needle at least until the butterfly needle is received by the butterfly needle insertion device.

30. The system of claim 23, wherein the butterfly needle retainer is a catch assembly disposed within the housing and providing an aperture through which at least a portion of the drive plunger moves, the catch assembly providing a pair of opposing catch springs disposed proximate to opposing sides of the drive plunger.

31. The system of claim 23, wherein the stop is provided by opposing buttons extending from sides of the housing, depression of the buttons releasing the drive plunger from its retracted state.

32. The system of claim 23, wherein the proximal end of the drive plunger has a concave seat structured and arranged to receive the needle hub of the butterfly needle.

33. The system of claim 23, wherein the opposing grooves are substantially perpendicular to the opposing channels.

34. A method for administering a butterfly needle to a patient, comprising:
providing a butterfly needle insertion device, comprising:
housing comprising a shell with a distal end and a proximal opening, the proximal opening having two opposing grooves each having a width to receive a wing of a butterfly needle, the opposing grooves extending from the proximal opening towards the distal end, and two opposing channels disposed between the opposing grooves, the opposing channels extending towards the distal end;
a drive plunger occupying at least a portion of the space defined by the shell of the housing and in shifting engagement with the housing, wherein the drive plunger has a distal end to be engaged by a stop to hold the drive plunger in a retracted position and a proximal end structured and arranged to receive the butterfly needle with the wings extending through the opposing grooves and beyond the housing, a supply line of the butterfly needle received by one of the opposing channels;
a butterfly needle retainer disposed within the housing and structured and arranged to temporarily hold the butterfly needle to the proximal end of the drive plunger when the drive plunger is in the retracted position prior to insertion of the butterfly needle into the patient;
a drive spring associated with the drive plunger such that when the drive plunger is driven into the housing to the retracted position, the drive spring is loaded and when the drive plunger is released, the drive plunger travels distally from the force of the drive spring unloading to deliver the butterfly needle into the patient;
providing an inserter base, the inserter base structured and arranged to receive the butterfly needle, the housing disposed over the inserter base and depressed down about the inserter base to retract the drive plunger within the housing and engage the butterfly needle to the butterfly needle retainer;
disposing the butterfly needle upon the inserter base;
disposing the housing over the inserter base;
depressing the housing down upon the inserter base to retract the drive plunger into the housing and engage the butterfly needle temporarily with the butterfly needle retainer;
disposing the proximal opening over an intended infusion site of the patient; and
releasing the stop to dispose the butterfly needle into the patient.
